(19) United States
(12) Patent Application Publication
(10) Pub. No.: US 2016/0296439 A1
(43) Pub. Date: Oct. 13, 2016

(54) COMPOSITION

(71) Applicant: CONOPCO, INC., D/B/A UNILEVER, Englewood Cliffs, NJ (US)

(72) Inventors: Abid IFTIKHAR, Liverpool (GB); Andrew Malcolm MURRAY, Parkgate, Cheshire (GB); Prem Kumar Cheyalazhagan PAUL, Spital, Wirral, Merseyside (GB)

(73) Assignee: Conopco, Inc., d/b/a UNILEVER, Englewood Cliffs, NJ (US)

(21) Appl. No.: 15/036,425

(22) PCT Filed: Nov. 19, 2014
(86) PCT No.: PCT/EP2014/074999

§ 371 Date: May 13, 2016

(30) Foreign Application Priority Data
Nov. 21, 2013 (EP) 13193933.2

(51) Int. Cl. A61K 8/365; A61Q 5/12; A45D 7/04; A61K 8/41; A61K 8/34; A61K 8/19; A61Q 5/06; A61K 8/04

(52) U.S. Cl. CPC A61K 8/365; A61Q 5/06; A61Q 5/12; A61K 8/042; A61K 8/416; A61K 8/342; A61K 8/19; A45D 7/04; A61K 2800/87

(57) ABSTRACT

Hair treatment composition comprising from 3 to 20% bi-or tri-carboxylic acid, from 0.1 to 10% wt. cationic surfactant and having a pH of from 1 to 2.9.

The present invention relates to a hair treatment composition and its use in styling hair.

Despite the prior art there remains a need for improved styling compositions.

Accordingly, the present invention relates to a composition comprising from 3 to 20% bi- or tri-carboxylic acid, from 0.2 to 10% wt. cationic surfactant and having a pH of from 1 to 2.9.

We have surprisingly found that a composition can be stable at low pH and comprising a cationic surfactant and a carboxylic acid.

Preferably, the hair treatment composition comprises a fatty material selected from fatty alcohols, fatty acids, fatty amides and mixtures thereof. Most preferably, the fatty material is fatty alcohol.

By fatty is meant having an alkyl chain with from 6 to 30 carbons, preferably from 10 to 22 carbons. Preferably, such an alkyl chain is saturated or unsaturated, branched or un-branched but it is most preferable that it is un-branched and saturated.

Preferably, the hair treatment composition comprises from 0.1 to 15% wt. fatty material.

Preferably, the cationic surfactant has the formula N⁺R¹R²R³R⁴ wherein R¹, R², R³ and R⁴ are independently (C₁ to C₃₀) alkyl or benzyl. Preferably, one, two or three of R¹, R², R³ and R⁴ are independently (C₄ to C₃₀) alkyl and the other R¹, R², R³ and R⁴ group or groups are (C₁-C₆) alkyl or benzyl. More preferably, one or two of R¹, R², R_{3 R}³ and R⁴ are independently (C₆ to C₃₀) alkyl and the other R¹, R², R³ and R⁴ groups are (C₁-C₆) alkyl or benzyl groups. Optionally, the alkyl groups may comprise one or more ester (—OCO— - or —COO—) and/or ether (—O—) linkages within the alkyl chain. Alkyl groups may optionally be substituted with one or more hydroxyl groups. Alkyl groups may be straight chain or branched and, for alkyl groups having 3 or more carbon atoms, cyclic. The alkyl groups may be saturated or may contain one or more carbon-carbon double bonds (eg, oleyl). Alkyl groups are optionally ethoxylated on the alkyl chain with one or more ethylenoxy groups.

Suitable cationic surfactants for use in conditioner compositions according to the invention include cetyltrimethylammonium chloride, behenyltrimethylammonium chloride, cetylpyridinium chloride, tetramethylammonium chloride, tetraethylammonium chloride, octyltrimethylammonium chloride, dodecyltrimethylammonium chloride, hexadecyltrimethylammonium chloride, octyldimethylbenzylammonium chloride, decyldimethylbenzylammonium chloride, stearyldimethylbenzylammonium chloride, didodecyldimethylammonium chloride, dioctadecyldimethylammonium chloride, tallowtrimethylammonium chloride, dihydrogenated tallow dimethyl ammonium chloride (eg, Arquad 2HT/75 from Akzo Nobel), cocotrimethylammonium chloride, PEG-2-oleammonium chloride and the corresponding hydroxides thereof. Mixtures of any of the foregoing materials may also be suitable. A particularly useful cationic surfactant for use in conditioners according to the invention is cetyltrimethylammonium chloride, available commercially, for example as GENAMIN CTAC, ex Hoechst Celanese.

Another particularly useful cationic surfactant for use in conditioners according to the invention is behenyltrimethylammonium chloride, available commercially, for example as GENAMIN KDMP, ex Clariant. Preferably, the cationic surfactant is a mono- or di-alkyl quaternary ammonium salt comprising from 8 to 24 carbons in the alkyl chain.

Preferably the cationic surfactant is present at from 0.1 to 10% wt., more preferably from 0.5 to 7.5% wt. and most preferably from 0.5 to 5% wt. of the treatment composition of the invention.

Preferably, the composition comprises from 0 to 0.1% wt. and is more preferably free from an amidoamine corresponding to the general formula (I):

R1 CONH(CH2)mN(R2)R3

in which R¹ is a hydrocarbyl chain having 10 or more carbon atoms, R² and R³ are independently selected from hydrocarbyl chains of from 1 to 10 carbon atoms, and m is an integer from 1 to about 10; and

Notable amidoamines include stearamido-propyldimethylamine, stearamidopropyldiethylamine, stearamidoethyldiethylamine, stearamidoethyldimethylamine, palmitamidopropyldimethylamine, palmitamidopropyl-diethylamine, palmitamidoethyldiethylamine, palmitamidoethyldimethylamine, behenamidopropyldimethyl-amine, behenamidopropyldiethylmine, behenamidoethyldiethyl-amine, behenamidoethyldimethylamine, arachidamidopropyl-dimethylamine, arachidamidopropyldiethylamine, arachid-amidoethyldiethylamine, arachidamidoethyldimethylamine, and mixtures thereof.

Such amidoamines are typically included with an acid which protonises the amine to form a cationic surfactant.

We have surprisingly found that such protonated amidoamine cationic surfactants are entirely unsuitable to the present invention and produce an unstable conditioning product. This was entirely unexpected as such materials are commonly used in rinse-off conditioning compositions.

Preferably, the carboxylic acid is selected from aconitic acid, tricarballylic acid, malonic acid, tartaric acid, citric acid and mixtures thereof. Most preferably, it is aconitic acid or citric acid.

Preferably, the hair treatment composition is a rinse-off composition.

Preferably, the hair treatment composition is a single-dose composition. By this is meant that it is to be applied to the hair in one go.

In a second aspect the invention relates to a method for styling hair comprising:
applying to hair a hair treatment composition as previously described;
leaving the composition on the hair for from 5 to 90 minutes;
rinsing the product from the hair; and
styling.

Preferably, the treatment composition is left on the hair for from 10 to 40 minutes and more preferably from 15 to 25 minutes.

Preferably, rinsing is with water and preferably the hair is styled, preferably straightened, before rinsing.

By styling, is preferably meant straightening or applying body.

Preferably, the method comprises applying the hair treatment composition to dry hair.

By ‘dry hair’ is meant that the amount of free water disposed on the cuticle has been substantially removed by towelling or evaporation such that it constitutes no more than 25% wt. of the hair fibre as a whole.

This means that the hair has not been washed or actively wetted, such as by shampooing, conditioning, rinsing or otherwise treating with an aqueous composition in the preceding 2, preferably 3 hours and has been permitted to acclimatise to atmospheric conditions.

In such circumstances there is substantially no free water on the hair fibre which interferes with the adsorption of the hair treatment composition on application. Preferably, the method according to the second aspect comprises applying the hair treatment composition at a temperature at from 15 to 40° C. Preferably, the treatment composition is applied to the hair at room temperature.

We have surprisingly found that the composition of the invention can be applied to the hair and provide a styling benefit without the use of hair irons.

Preferably, the method comprises applying the hair treatment composition at a relative humidity of from 5 to 95% at 25° C.

In a third aspect there is provided a method for durable styling to the hair by conducting the method according to the second aspect and then washing the hair. In such circumstances we have found that the styling benefit applied to the hair is durable despite the hair being subsequently washed. The durable nature of the styling is maintained for up to 5, preferably, 10 and most preferably 15 washes.

By styling is preferably meant straightening.

One would ordinarily expect a hair style to be significantly diminished by a shampooing step.

Preferably, more than 50% of the style is maintained after 3 washes.

In a fourth aspect there is provided a hair treatment composition according to the first aspect obtainable by forming a solution of citric acid, adjusting the pH such that the pH of the solution is from 1 to 2.9, preferably from 1.0 to 2.9 and most preferably from 2.2 to 2.8, separately forming a composition comprising the cationic surfactant and any fatty material and any further ingredients before then adding the buffered citric acid solution.

In a fifth aspect there is provided a packaged product comprising from 100 to 300 ml of the hair treatment composition. Preferably, the product comprises 150 to 250 ml of the hair treatment composition.

In a sixth aspect there is provided a method for styling hair comprising applying from 3 to 20 g, preferably from 5 to 18 g and most preferably from 8 to 16 g citric acid to the hair.

Preferably, the citric acid is applied as an aqueous solution.

### EXAMPLES

The following working example formulations.

The citric acid is made separately and buffered to a pH of 2.2-2.3 with sodium hydroxide before adding to a conditioning composition base comprising the remaining materials, notably the cationic surfactant and fatty alcohol which together form the conditioning gel phase.

| Trade Name | % w | % w | % w | % w |
|---|---|---|---|---|
| Water | To 100 | To 100 | To 100 | To 100 |
| Natrosol | — | — | — | 0.1 |
| Behentrimmonium chloride (70%) | 4.3 | 0.5714 | 1.1428 | 0.5714 |
| Cetearyl alcohol | 6 | 1.5 | 3 | 1.5 |
| Dimethylacrylamide/ | — | — | — | 1.5 |
| Ethyltrimonium Chloride | | | | |
| Methacrylate Copolymer | | | | |
| (and) Propylene Glycol | | | | |
| Dicaprylate/Dicaprate | | | | |
| (and) PPG-1 Trideceth-6 | | | | |
| (and) C10-C11 Isoparaffin* | | | | |
| Aminosilicone (70%) | 1.428 | 1.428 | 1.428 | 1.428 |
| Preservative (70%) | 0.35 | 0.35 | 0.35 | 0.35 |
| Perfume | 0.6 | 0.6 | 0.6 | 0.6 |
| Citric acid (25% wt aq) | 20 | 20 | 20 | 20 |
| NaOH (50%) | 0.91 | 0.91 | 0.91 | 0.91 |
| EDTA | 0.05 | 0.05 | 0.05 | 0.05 |

*Tinovis CD ex BASF

The following data supports a finding that the straightening achieved through applying to the hair a hair treatment composition as described in the above table is durable through multiple washes.

| | | | | | |
|---|---|---|---|---|---|
| Stage | A | B | C | D | E |

Volume [mm²]

| | | | | | |
|---|---|---|---|---|---|
| Before | 15474.73 | 17214.53 | 16720.19 | 17085.74 | 16283.36 |
| treatment | | | | | |
| After | 14914.97 | 10919.82 | 10791.08 | 11439.89 | 11541.37 |
| treatment | | | | | |
| AW1 | 14978.74 | 10860.80 | 12622.58 | 12480.31 | 13421.44 |
| AW3 | 14848.99 | 11538.96 | 13427.67 | 13212.13 | 14161.78 |
| AW5 | 14882.07 | 11278.24 | 13206.93 | 13375.39 | 13621.05 |
| AW10 | 14886.58 | 11838.58 | 12364.28 | 13224.90 | 14097.57 |
| AW15 | 13927.47 | 11188.64 | 12216.97 | 13636.92 | 14520.90 |
| AW20 | 14218.68 | 13249.26 | 15034.80 | 15348.22 | 15422.16 |

Percentage Volume

| | | | | | |
|---|---|---|---|---|---|
| Before | 100 | 100 | 100 | 100 | 100 |
| treatment | | | | | |
| After | 96.38 | 63.43 | 64.54 | 66.96 | 70.88 |
| treatment | | | | | |
| AW1 | 96.79 | 63.09 | 75.49 | 73.05 | 82.42 |
| AW3 | 95.96 | 67.03 | 80.31 | 77.33 | 86.97 |
| AW5 | 96.17 | 65.52 | 78.99 | 78.28 | 83.65 |
| AW10 | 96.20 | 68.77 | 73.95 | 77.40 | 86.58 |
| AW15 | 90.00 | 65.00 | 73.07 | 79.81 | 89.18 |
| AW20 | 91.88 | 76.97 | 89.92 | 89.83 | 94.71 |

‘AW X’ means after X washes.

The following comparative examples were also assessed for suitability.

| INCI Name | Active (%) |
|---|---|
| Water | To 100 |
| Cetearyl Alcohol | 4.00 |
| Stearamidopropyl Dimethylamine | 1.00 |
| Behentrimonium Chloride | 0.70 |
| Lactic Acid | 0.28 |
| Citric acid premix | 20.00 |
| Neutraliser | 1.8291 |
| Arginine | 0.30 |
| Disodium EDTA | 0.05 |
| Silicone | 1.23 |
| Fragrance | 0.30 |
| Preservative | 0.245 |

Citric acid premix (pH 2.2-2.3)

| | |
|---|---|
| Citric acid | 5.00 |
| NaOH | 0.49 |
| Emulsifier | 0.05 |
| Water | 14.00 |

The above formulation was unstable.

Styling Creme

| Chemical name (order of addition) | % wt. |
|---|---|
| Citric acid (25% aq) | 20.00 |
| NaOH | 0.49 |
| Versene NA2 | 0.05 |
| Water | 14.00 |
| Cetyl Hydroxyethylcellulose | 0.10 |
| Cetearyl Alcohol 30-70 C16-C18 | 2.50 |
| White Mineral Oil 70 Sus | 1.00 |
| Stearamidopropyl Dimethylamine | 0.70 |
| Lactic Acid | 0.26 |
| Dimethicone and Trideceth-5 | 0.33 |
| Glycerin | 1.00 |
| Cyclopentasiloxane and Trimethylsiloxysilicate | 0.60 |
| and Dimethiconol | |
| Parfum | 0.40 |
| DMDM Hydantoin (and) | 0.35 |
| Iodopropynyl Butylcarbamate | |
| Water | To 100 |

The citric acid was formed as a premix which is added to the remaining ingredients at the last stage.

The formulation is unstable.

1. Packaged hair treatment comprising from 50 to 300 ml hair treatment composition comprising from 3 to 20% bi- or tri-carboxylic acid, from 0.1 to 10% wt. cationic surfactant and having a pH of from 1 to 2.9, wherein the composition comprises a fatty material selected from fatty alcohols, fatty acids, fatty amides and mixtures thereof and wherein the cationic surfactant has the formula N⁺R¹R²R³R⁴ wherein R′, R², R³ and R⁴ are independently (C₁ to C₃₀) alkyl or benzyl, and wherein the hair treatment composition is a single dose.

2. Packaged hair treatment product according to claim 1 comprising from 0.1 to 15% wt. fatty material.

3. Packaged hair treatment product according to claim 1 comprising fatty alcohol.

4. Packaged hair treatment product according to claim 1 wherein the cationic surfactant is a mono- or di-alkyl quaternary ammonium salt comprising from 8 to 24 carbons in the alkyl chain.

5. Packaged hair treatment product according to claim 1 wherein the carboxylic acid is selected from aconitic acid, tricarballylic acid, malonic acid, tartaric acid, citric acid and mixtures thereof.

6. Packaged hair treatment product according to claim 1 obtainable by forming a conditioning composition comprising a cationic surfactant and a fatty material and adding a solution of citric acid which has been buffered to a pH of from 1 to 2.8.

7. Packaged hair treatment product according to claim 6 wherein the conditioning composition is formed by heating cationic surfactant in water and then adding fatty material at a temperature greater than the melting temperature of the fatty material and then adding a solution of citric acid buffered to a pH of from 1.0 to 2.9.

8. Packaged hair treatment product according to claim 1 comprising from 100 to 200 ml hair treatment composition.

9. A method for styling hair comprising:
applying to hair a hair treatment composition comprising from 3 to 20% bi- or tri-carboxylic acid, from 0.1 to 10% wt. cationic surfactant and having a pH of from 1 to 2.9, wherein the composition comprises a fatty material selected from fatty alcohols, fatty acids, fatty amides and mixtures thereof and wherein the cationic surfactant has the formula N³⁰R¹R²R³R⁴ wherein R¹, R², R³ and R⁴ are independently (C₁ to C₃₀) alkyl or benzyl.
leaving the composition on the hair for from 5 to 90 minutes;
rinsing the product from the hair; and
styling
wherein the treatment is applied to dry hair.

10. Method according to claim 9 of durably styling the hair by applying to the hair a hair treatment composition according to claim 1.
